(19) United States
(12) Patent Application Publication
(10) Pub. No.: US 2007/0259794 A1
(43) Pub. Date: Nov. 8, 2007

(54) Shampoo Composition

(76) Inventor: Richard Wachsberg, Toronto (CA)

Correspondence Address: SYNNESTVEDT LECHNER & WOODBRIDGE LLP, P O BOX 592, 112 NASSAU STREET, PRINCETON, NJ, 08542-0592, US

(21) Appl. No.: 11/381,758

(22) Filed: May 5, 2006

(51) Int. Cl. A61K 8/00

(52) U.S. Cl. 510/119

(57) ABSTRACT

A shampoo composition is disclosed which comprises one or more surfactants, suspending agents, an anti-dandruff agent that preferably comprises ketoconazole, and an antioxidant for preventing oxidation of the anti-dandruff agent. The antioxidant is selected and present in sufficient quantity to preserve at least 90% of the activity of the anti-dandruff agent under conditions of accelerated stability testing for at least 12 weeks.

### FIELD OF THE INVENTION

The present invention relates to shampoo compositions containing anti-dandruff ingredients and an antioxidant preservative agent for storage stability of the composition.

### BACKGROUND AND PRIOR ART

Shampoo compositions are well known and widely published in the patent and technical literature and typically contain surfactants, co-polymers or other suspension agents, anti-dandruff agents such as ketoconazole, and optionally other constituents. For example, U.S. Pat. No. 6,706,258 to Gallagher et al. describes shampoo compositions containing cleansing surfactants, polymers, and other constituents. Typically, a shampoo comprises an emulsion or micro-emulsion, and includes suspending agents to prevent settling and separation of the solids.

Optional ingredients include scent and colouring constituents, such as approved dyes.

The anti-dandruff agent ketoconazole is an anti-microbial compound suitable for topical application in shampoos. However, ketoconazole has limited shelf life and therefore requires use of a preservative such as an antioxidant. However, prior art shampoo compositions have not provided preservatives that provide wholly satisfactory long term storage stability.

### SUMMARY OF THE INVENTION

One of the objects of the present invention is to provide an anti-dandruff shampoo, having a relatively long shelf life with minimal degradation, discolouration, or other undesired changes to the composition as a whole, or the anti-dandruff agent or agents in particular.

It is a further object to provide an anti-dandruff shampoo containing an active anti-dandruff agent and an antioxidant preservative for storage stability of the shampoo.

It is a further object to provide an anti-dandruff shampoo containing ketoconazole as the active anti-dandruff agent, and the compound tetradibutyl pentaerithrityl hydroxyhydrocinnamate (TPH) as an antioxidant preservative.

According to one aspect, the invention comprises a shampoo composition comprising at least one anionic surfactant, a suspending agent, an anti-dandruff agent, and an antioxidant in which the antioxidant preserves at least 90% and preferably at least about 99% of the activity of the anti-dandruff agent under conditions of accelerated stability testing for at least 12 weeks.

According to another aspect, the invention comprises a shampoo composition comprising at least one anionic surfactant, at least one suspending agent such as a polymeric compound, ketoconazole in an amount effective for providing anti-dandruff activity, and an effective amount of TPH.

According to a further aspect, the anionic surfactants are present in a concentration range of from about 1% to 60%, preferably from 10% to 50%, more preferably from 20% to 45%, and most preferably from 25% to 40%, by weight of the shampoo composition. The anionic surfactant may comprise one or more of sodium laureth sulphate, sodium lauryl sulphate, ammonium laureth sulphate, or ammonium lauryl sulphate. The polymeric component preferably comprises an acrylate co-polymer, in a concentration range from about 1% to 20%, preferably from 2% to 15%, more preferably from 3% to 10%, and most preferably from 4% to 8%, by weight.

The anti-dandruff agent preferably comprises ketoconazole in a concentration range from about 0.07% to 3%, preferably from 0.05% to 2.5%, more preferably from 0.5% to 2%, and most preferably 0.75% to 1.25%, by weight.

According to another aspect, the antioxidant consists of one or more of PTH, BHA, TBHQa, tocopherol (comprising one or more tocopherol compounds, for example tocopherol acetate), acetate, and tetrabutyl pentaerithrityl hydroxycinnamate. Preferred antioxidants are TPH, BHA, and tocopherols. The most preferred antioxidant is TPH. The suspending agent may comprise an acrylate co-polymer such as Carbomer Aqua SF-1™ or Viscolam MAC 10™ or a combination thereof. TPH preferably is present in a concentration range from about 0.001% to 1%, preferably from 0.005% to 0.5%, and most preferably from 0.05% to 0.3%, by weight.

The composition may further comprise one or more of acetate, fragrance, water, and colouring. The compositions may also comprise other known shampoo constituents, described in more detail below.

All quantities set forth herein may depart from the expressed amount by 0% to 10%. It will be understood that terminology such as “about” or “approximately” relates to departures within this range, although such departures or variations may be present whether or not such terminology is employed, unless it is specifically stated that a particular quantity may not be thus varied.

It will be further understood that chemical constituents described herein may also include their close chemical equivalents, such as salts and other chemical equivalents.

### DETAILED DESCRIPTION

Shampoo compositions according to the invention comprise the following components:

1. Anionic Surfactants

Suitable surfactants include but are not limited to sodium laureth sulphate, sodium lauryl sulphate, ammonium laureth sulphate, ammonium lauryl sulphate, or any combination of said surfactants.

2. Polymer Compounds

A preferred polymer comprises an acrylates co-polymer, including but not limited to Carbomer Aqua SF-1™ or Viscolam MAC 10™. Mixtures of polymers are also contemplated.

3. Anti-Dandruff Compound

The preferred anti-dandruff compound is ketoconazole.

4. Antioxidant

The preferred antioxidant is tetradibutyl pentaerithrityl hydroxyhydrocinnamate. Other antioxidants include but are not limited to BHA, TBHQ, tocopherol (comprising one or more of the tocopherols), tetradibutyl pentaerithrityl hydroxyhydrocinnamate, tocopheryl acetate, or combinations thereof.

The antioxidant TPH is sold commercially under the trade-mark Tinogard TT™, by Ciba Specialty Chemicals.

### EXAMPLE 1

### Reference Product

A shampoo composition comprising the constituents identified in Table 1 below comprises a reference product:

TABLE 1

| | | | 500 gr | | |
|---|---|---|---|---|---|
| | | Ingredients (INCI) | Item Number | % | Grams |
| | 1 | DI Water | | 32.429040 | 182.145200 |
| | 2 | Carbopol Aqua SF-1 | | 5.000000 | 25.000000 |
| | 3 | Sodium Laureth Sulfate | | 35.000000 | 175.000000 |
| | 4 | NaOH 50 | | 0.400000 | 2.000000 |
| | 5 | Tetrasodium EDTA | | 0.050000 | 0.250000 |
| | 6 | Polyquaternium-7 | | 1.250000 | 6.250000 |
| | 7 | Cocamide MEA | | 2.000000 | 10.000000 |
| | 8 | Glycol Distearate | | 2.000000 | 10.000000 |
| | 9 | Sodium Cocoyl Sarcosinate | | 10.000000 | 50.000000 |
| | 10 | Fragrance Aroma Tech 5053143 | | 0.220000 | 1.100000 |
| | 11 | DI Water | | 0.100000 | 0.500000 |
| → | 12 | Tinogard TL | | 0.100000 | 0.500000 |
| | 13 | Quaternium-15 | | 0.200000 | 1.000000 |
| | 14 | DI Water | | 0.500000 | 2.500000 |
| | 15 | Ketoconazole | | 1.000000 | 5.000000 |
| | 16 | DI Water | | 8.000000 | 30.000000 |
| | 17 | FD & C Bluee 1 | | 0.000520 | 0.002600 |
| | 18 | DI Water | | 0.500000 | 2.500000 |
| | 19 | FD & C Yellow 5 | | 0.000210 | 0.001050 |
| | 20 | DI Water | | 0.500000 | 2.500000 |
| | 21 | D & C Red 33 | | 0.000230 | 0.001150 |
| | 22 | DI Water | | 0.500000 | 2.500000 |
| | 23 | Sodium Chloride | | 2.250000 | 11.250000 |
| | | | Total | | |

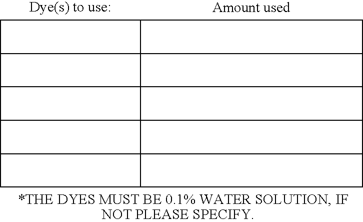

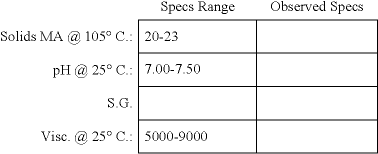

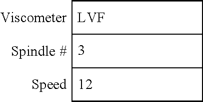

### EXAMPLE 2

A shampoo composition comprising the following constituents:

TABLE 2

| | | 800 gr | | |
|---|---|---|---|---|
| | Ingredients (INCI) | Item Number | % | Grams |
| 1 | DI Water | | 32.429040 | 259.432320 |
| 2 | Carbopol Aqua SF1 | | 5.000000 | 40.000000 |
| 3 | Sodium Laureth Sulfate | | 35.000000 | 280.000000 |
| 4 | NaOH 50% Solution | | 0.400000 | 3.200000 |
| 5 | Tetrasodium EDTA | | 0.050000 | 0.100000 |
| 6 | Polyquaternium-7 | | 1.250000 | 10.000000 |
| 7 | Cocamide MEA | | 2.000000 | 16.000000 |
| 8 | Glycol Distearate | | 2.000000 | 16.000000 |
| 9 | Sodium Cocyl Sarcosinate (Crodasinic CS 30) | | 10.000000 | 60.000000 |
| 10 | Fragrance Aroma Tech 5053143 | | 0.220000 | 1.760000 |
| 11 | Tinogard TT | | 0.100000 | 0.800000 |
| 12 | Tinogard TL | | 0.100000 | 0.800000 |
| 13 | Quaternium-15 | | 0.200000 | 1.600000 |
| 14 | DI Water | | 0.500000 | 4.000000 |
| 15 | Ketoconazole | | 1.000000 | 8.000000 |
| 16 | DI Water | | 6.000000 | 48.000000 |
| 17 | FD & C Blue 1 | | 0.000520 | 0.004150 |
| 18 | DI Water | | 0.500000 | 4.000000 |
| 19 | FD & C Yellow 5 | | 0.000210 | 0.001680 |
| 20 | DI Water | | 0.500000 | 4.000000 |
| 21 | D & C Red 33 | | 0.000230 | 0.001840 |
| 22 | DI Water | | 0.500000 | 4.000000 |
| 23 | Sodium Chlooride | | 2.250000 | 18.000000 |
| | | Total % | | |

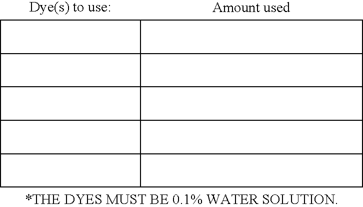

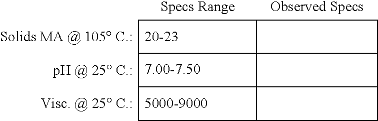

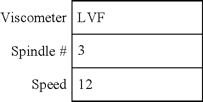

### EXAMPLE 3

TABLE 3

| | | | 800 gr | | |
|---|---|---|---|---|---|
| | | Ingredients (INCI) | Item Number | % | Grams |
| | 1 | DI Water | | 32.429040 | 259.432320 |
| | 2 | Carbopol Aqua SF-1 | | 5.000000 | 40.000000 |
| | 3 | Sodium Laureth Sulfate | | 35.000000 | 280.000000 |
| | 4 | NaOH 50% solution | | 0.400000 | 3.2000000 |
| | 5 | Tetrasodium EDTA | | 0.050000 | 0.400000 |
| | 6 | Polyquaternium-7 | | 1.250000 | 10.000000 |
| | 7 | Cocamide MEA | | 2.000000 | 16.000000 |
| | 8 | Glycol Distearate | | 2.000000 | 16.000000 |
| | 9 | Sodium Cocoyl Sarcosinate | | 10.000000 | 80.000000 |
| | 10 | Fragrance AromaTech 5053143 | | 0.220000 | 1.760000 |
| → | 11 | Tocopherols | | 0.100000 | 0.800000 |
| | 12 | Tinogard TL | | 0.100000 | 0.800000 |
| | 13 | Quaternium-15 | | 0.200000 | 1.600000 |
| | 14 | DI Water | | 0.500000 | 4.000000 |
| | 15 | Ketoconazole | | 1.000000 | 8.000000 |
| | 16 | DI Water | | 5.000000 | 48.000000 |
| | 17 | FD & C Blue 1 | | 0.000520 | 0.004180 |
| | 18 | DI Water | | 0.500000 | 4.000000 |
| | 19 | FD & C Yellow 5 | | 0.000210 | 0.001680 |
| | 20 | DI Water | | 0.500000 | 4.000000 |
| | 21 | D & C Red 33 | | 0.000230 | 0.001840 |
| | 22 | DI Water | | 0.500000 | 4.000000 |
| | 23 | Sodium Chloride | | 2.250000 | 16.000000 |
| | | | Total % | | |

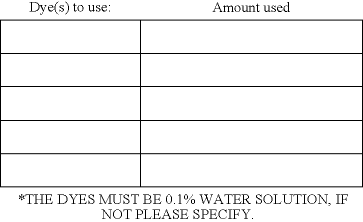

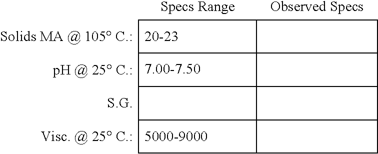

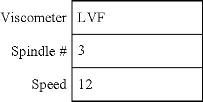

### EXAMPLE 4

TABLE 4

| | | | 800 gr | | |
|---|---|---|---|---|---|
| | | Ingredients (INCI) | Item Number | % | Grams |
| | 1 | DI Water | | 32.429040 | 259.432320 |
| | 2 | Carbopol Aqua SF-1 | | 5.000000 | 40.000000 |
| | 3 | Sodium Laureth Sulfate | | 35.000000 | 280.000000 |
| | 4 | NaOH 50% solution | | 0.400000 | 3.200000 |
| | 5 | Tetrasodium EDTA | | 0.050000 | 0.400000 |
| | 6 | Polyquaternium-7 | | 1.250000 | 10.000000 |
| | 7 | Cocamide MEA | | 2.000000 | 16.000000 |
| | 8 | Glycol Distearate | | 2.000000 | 16.000000 |
| | 9 | Sodium Cocoyl Sarcosinate (Crodasinic CS 30) | | 10.000000 | 80.000000 |
| | 10 | Fragrnace Aromatech 6053143 | | 0.220000 | 1.760000 |
| → | 11 | BHA | | 0.100000 | 0.800000 |
| | 12 | Tinogard TL | | 0.100000 | 0.800000 |
| | 13 | Quaternium-15 | | 0.200000 | 1.600000 |
| | 14 | DI Water | | 0.500000 | 4.000000 |
| | 15 | Ketoconazole | | 1.000000 | 8.000000 |
| | 16 | DI Water | | 6.000000 | 48.000000 |
| | 17 | FD & C Blue 1 | | 0.000520 | 0.004160 |
| | 18 | DI Water | | 0.500000 | 4.000000 |
| | 19 | FD & C Yellow 5 | | 0.000210 | 0.001680 |
| | 20 | DI Water | | 0.500000 | 4.000000 |
| | 21 | D & C Red 33 | | 0.000230 | 0.001840 |
| | 22 | DI Water | | 0.500000 | 4.000000 |
| | 23 | Sodium Chloride | | 2.250000 | 18.000000 |
| | | | Total % | | |

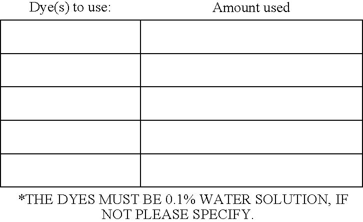

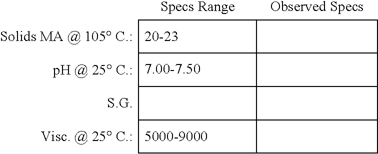

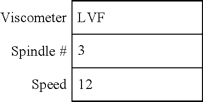

In the above examples, the listed percentage of each component relates to the percentage by weight of the overall composition.

In the composition of the above Examples, the pH at 25° is between 7.0 and 7.5. Viscosity at 25° C. was 5000 to 9000 CPS, and at 20 to 23%.

The compositions of the above examples are prepared by combining ingredients in the order listed in the above charts. Components 1 to 6 are added at ambient temperature, while mixing the formulation. Components 7, 8, and 9 are added when the mixture is elevated to a temperature of between 80° C. to 85° C., following which the mixture is cooled.

Ingredients 10, 11, and 12 are pre-dissolved in a tertiary tank, prior to mixture with the other components of the composition. The same procedure is followed with components 13 and 14, and 15 and 16, namely pre-mixture followed by combining with the main mixture. Colour and viscosity are then adjusted with components 17 to 23, to yield the shampoo compositions of examples 1 through 4.

Accelerated stability testing carried out on the products of examples 1 through 4 was carried out according to the steps described below, consisting generally of 12 weeks at elevated and reduced temperatures, following which measurements were conducted of viscosity and percentage of ketoconazole remaining the composition.

Protocol For Accelerated Stability Testing

1. Keep a minimum of six 2 oz samples for stability testing.

2. Allow the sample to mature for up to four days prior to testing.

3. Record the starting specs for the sample. This includes pH, solids and viscosity.

4. Perform stability testing as defined below in the subsections.

5. Place a sample in freeze/thaw, room temperature, 37° C., 45° C., window and fluorescent light box if applicable. All samples except freeze/thaw are examined weekly for four weeks thereafter at six, eight, and twelve weeks.

6. For the freeze/thaw test leave sample in the freezer at least twenty-four hours.

7. Remove sample from the freezer and store for twenty-four hours at room temperature.

8. Examine for separation, thinning, and thickening.

9. If there is no separation, return sample to the freezer and repeat steps 7 to 9 three times.

The results of the stability testing performed on the examples are summarized in table 5:

TABLE 5

| Example | Initial ph | Viscosity | % Ketoconazole | pH | Viscosity | % Ketoconazole | Anitoxidant |
|---|---|---|---|---|---|---|---|
| 1 | 7.25 | 7500 | 1% | 7.21 | 7300 | 0.856 | Non |
| 2 | 7.2 | 7300 | 1% | 7.16 | 7600 | 0.997 | TPH |
| 3 | 7.18 | 7000 | 1% | 7.26 | 7300 | 0.903 | Tocopherols |
| 4 | 7.36 | 8200 | 1% | 7.31 | 8700 | 0.908 | BHA |

Although the present invention has been described in part by way of detailed descriptions of preferred embodiments thereof, it will be apparent to those skilled in the relevant art that departures from and variations to the embodiments described herein may be made without departing from the full scope of the invention. The full scope of the present invention is defined more particularly in the claims to this patent specification, including all elements, quantities, and the like that may reasonably be considered equivalents of the like elements set forth in the present claims.

1. A shampoo composition comprising at least one anionic surfactant, a suspending agent, an anti-dandruff agent, and an antioxidant in a quantity sufficient to preserve at least 90% of the activity of said anti-dandruff agent under conditions of accelerated stability testing for at least 12 weeks.

2. The composition of claim 1 wherein said antioxidant comprises one or more of BHA, a tocopherol and TPH (tetradibutyl pentaerithrityl hdroxyhydrocinnamate).

3. The composition of claim 2 wherein said antioxidant comprises TPH.

4. The composition of claim 1, wherein said suspending agent comprises an acrylate co-polymer.

5. The composition of claim 4, wherein said acrylate co-polymer comprises one or both of Carbomer Aqua SF-1™ or Viscolam MAC 10™.

6. The composition of claim 1, wherein said anti-dandruff agent comprises ketoconazole.

7. The composition of claim 1, further comprising one or more of acetate, fragrance, water, and colouring.

8. The composition of claim 6, wherein said ketoconazole is present in an amount of between 0.07% and 3%.

9. The composition of claim 6, wherein said ketoconazole is present in an amount of between 0.05% and 2.5%.

10. The composition of claim 6, wherein said ketoconazole is present in an amount between 0.75% and 1.25%, by weight.

11. The composition of claim 3, wherein said TPH is present in an amount between 0.001% and 1.0%, by weight.

12. The composition of claim 3, wherein said TPH is present in an amount between 0.005% and 0.5%, by weight.

13. The composition of claim 3, wherein said TPH is present in an amount between 0.05% and 0.3%, by weight.

14. The composition of claim 3, wherein said TPH is present in an amount of about 0.1%, by weight.

15. The composition of claim 3 wherein said TPH is present in an amount sufficient to preserve at least 99% of the activity of said anti-dandruff agent under conditions of accelerated stability testing for at least 12 weeks.

16. A shampoo composition comprising at least one anionic surfactant, a suspending agent, ketoconazole, and TPH (tetradibutyl pentaerithrityl hdroxyhydrocinnamate).

17. The composition of claim 16 further comprising one or more of BHA and tocopherol.

18. The composition of claim 16, wherein said ketoconazole is present in an amount of between 0.07% and 3%.

19. The composition of claim 16, wherein said ketoconazole is present in an amount of between 0.05% and 2.5%.

20. The composition of claim 16, wherein said ketoconazole is present in an amount between 0.75% and 1.25%, by weight.

21. The composition of claim 16, wherein said TPH is present in an amount between 0.001% and 1.0%, by weight.

22. The composition of claim 16, wherein said TPH is present in an amount between 0.005% and 0.5%, by weight.

23. The composition of claim 16, wherein said TPH is present in an amount between 0.05% and 0.3%, by weight.

24. The composition of claim 16, wherein said TPH is present in an amount of about 0.1%, by weight.

25. The composition of claim 16 wherein said TPH is present in an amount sufficient to preserve at least 99% of the activity of said anti-dandruff agent under conditions of accelerated stability testing for at least 12 weeks.
